(19) United States
(12) Patent Application Publication
(10) Pub. No.: US 2015/0374611 A1
(43) Pub. Date: Dec. 31, 2015

(54) COMPOSITION FOR REDUCING THE TIME NEEDED TO DRY WET HAIR

(71) Applicant: KENRA PROFESSIONAL, LLC., Indianapolis, IN (US)

(72) Inventor: Thomas A. Lehman, JR., Indianapolis, IN (US)

(73) Assignee: KENRA PROFESSIONAL, LLC, Indianapolis, IN (US)

(21) Appl. No.: 14/844,667

(22) Filed: Sep. 3, 2015

Related U.S. Application Data
(62) Division of application No. 13/097,773, filed on Apr. 29, 2011.

(51) Int. Cl. A61K 8/892; A61K 8/97; A61K 8/81; A61Q 5/12; A61K 8/58; A61K 8/891

(52) U.S. Cl. CPC A61K 8/892; A61K 8/585; A61K 8/891; A61K 8/8164; A61Q 5/12; A61K 8/97

(57) ABSTRACT

A cosmetic composition useful to reduce the drying time of wet hair is disclosed. The composition includes a nonaqueous combination of a volatile silicone compound, such as cyclopentasiloxane, and a mixture of one or more silicone elastomers. The silicone elastomers may include, but are not limited to, dimethicone, cetearyl dimethicone crosspolymers, dimethicone crosspolymers, dimethicone/vinyl dimethicone crosspolymers, and dimethiconol.

### FIELD OF THE INVENTION

The present disclosure relates to compositions for use in hair that are capable of reducing the time needed to blow dry wet hair. In addition, the compositions may include those that thermally protect, reduce frizz, and/or soften hair and leave-in treatments that can be applied to hair as a spray.

### SUMMARY

One embodiment relates to a cosmetic composition useful to reduce the drying time of wet hair. The composition includes a nonaqueous combination of a volatile silicone compound, such as cyclopentasiloxane, and a mixture of one or more silicone elastomers. The silicone elastomers may include, but are not limited to, dimethicone, cetearyl dimethicone crosspolymers, dimethicone crosspolymers, dimethicone/vinyl dimethicone crosspolymers, and dimethiconol.

### DESCRIPTION OF THE DRAWINGS

FIG. 1 is a graphical representation of the percent of water lost in Samples A-J as a function of time.

### DETAILED DESCRIPTION

The composition includes a combination of silicone compounds. The composition can be applied with a spray bottle or other apparatus capable of spraying, misting, or otherwise applying in very small droplets, the composition onto a user's hair. The composition may also be applied as a gel or in other suitable forms. The composition may include small amounts of water, less than about one percent, but is nonaqueous. For the purposes of this disclosure, compositions that include less than about one percent water are considered to be nonaqueous. The composition includes at least one volatile silicone compound and at least one, but preferably a combination of at least two, silicone elastomers. The composition may also optionally include thermal protective agents, conditioning agents, and perfumes. The composition may be preferably less dense than water, with a preferred density of between 0.7 g/ml and 0.98 g/ml and viscosity between 0.0 cPs and 1500 cPs. It is noted that the viscosity may be measured using the RVT model viscometer, available from Brookfield Engineering Laboratories. The composition may also be preferably colorless.

The composition includes at least one volatile silicone compound, such as cyclopentasiloxane (D5), cyclomethicone (D4), and/or a substituted or an unsubstituted methyl silsesquioxanes. Suitable volatile silicone compounds include XIAMETER® PMX-0245 CYCLOPENTASILOXANE (formerly DC 245) from Dow Corning. Other suitable cyclopentasiloxanes may be available from Wacker, ISP, Rhodia, Jeen Corp., and Momentive Performance Materials. As used herein, the term “volatile” when employed in relation to a silicone compound includes compounds that exhibit a vapor pressure of more than about 0.2 mm Hg at 25° C. at one atmosphere of pressure or have a boiling point at one atmosphere of less than the boiling point of water, i.e. less than about 100° C.

Examples of suitable cyclomethicones may include Dow Corning 200 Fluid, Dow Corning 244 Fluid, Dow Corning 344 Fluid, and Dow Corning 345 Fluid (each commercially available from Dow Corning Corp.); SF-1204 and SF-1202 Silicone Fluids (each commercially available from G. E. Silicones), GE 7207 and 7158 (each commercially available from General Electric Co.); and SWS-03314 (each commercially available from SWS Silicones Corp.).

Examples of suitable methyl silsesquioxanes may include TMF 1.5 fluid (commercially available from Shin-Etsu Chemical Co), phenyl substituted silsesquioxanes (commercially available from Clariant as Silcare 15M60), n-Octyl substituted silsesquioxanes (commercially available as Silcare 31M60 and 31M50), hexyl methicone, caprylyl methicone, and lauryl methicone (each commercially available from Clariant as Silcare 41M10, 41M15 and 41M20, respectively).

The composition includes from about 75 to about 99 percent by weight of the final composition of one or more volatile silicone compounds, from about 80 to about 95 percent by weight of one or more volatile silicone compounds, or from about 85 to about 95 percent by weight of one or more volatile silicone compounds. All weight percentages disclosed herein are based upon the weight of the final composition, unless otherwise stated. In certain embodiments, the volatile silicone compound may be cyclopentasiloxane.

The composition also includes at least one silicone elastomer. Broadly, uncoated forms of cosmetically acceptable silicone elastomers may be used in the composition. These elastomers are considered non-emulsifying because they do not contain any appreciable amount of polyoxyalkylenes. Uncoated silicone elastomers include cross-linked or partially cross-linked cyclomethicone (and) dimethicone crosspolymers. Commercially available examples of suitable silicone elastomers include DC-9040, DC-9041 and DC-9045 (available from Dow Corning Inc), KSG-15 and USG-103 (available from Shin-Etsu Silicones of America), and GE 1229 (available from General Electric Silicones, Waterford, N. Y.).

In one embodiment, the composition includes a combination of silicone elastomers. Specifically, the composition may include from about 0.10 to about 10 percent by weight of a mixture of dimethicone and a cetearyl dimethicone crosspolymer, and preferably from about 0.25 to about 5 percent of the mixture. One specific example of a suitable silicone elastomer is DC 9041, commercially available from Dow Corning.

The composition may optionally include from about 0.25 to about 10 percent of a second silicone elastomer. The second elastomer may include a mixture of cyclopentasiloxane, dimethicone crosspolymer, dimethicone/vinyl dimethicone crosspolymer, and dimethiconol. Such mixture is commercially available under the trade name DC 9546, available from Dow Corning. Preferably, the composition includes from about 3 percent to about 5 percent of that mixture.

The composition may also optionally include other cosmetically acceptable additives, such as thermal protective agents (a.k.a. thermoprotective agent), hair conditioning agents, fragrance, and essential oils. Suitable oils may include, but are not limited to, agar, argan, balsam, basil, bay, bergamot, cardamom seed, cedarwood, cranberry, frankincense, geranium, grapefruit, jasmine, jojoba seed, lavender, lemon, litsea cubeba, orange, orris, parsley, patchouli, rose, rosemary, rosewood, sassafras, savory oil, star anise, tangerine oils, and combinations thereof.

In one embodiment, the composition may optionally include one or more of (1) from about 0.01 to about 1.0 percent by weight, preferably about 0.1 to about 0.3 percent by weight, argan oil, (2) from about 0.01 to about 1.0 percent by weight, preferably about 0.1 to about 0.3 percent by weight, jojoba seed oil, and (3) from about 0.1 to about 2.0 percent by weight, preferably 0.2 to about 0.5 percent by weight, of a fragrance additive.

In one embodiment, the composition may include at least one thermal protective agent, such as a sodium laneth 40 maleate/styrene sulfonate copolymer, or other suitable thermal protector. One commercially available thermal protective agent is Mirustyle X-HP from Croda. Another example is commercially available as Mirustyle X-HV from Croda.

In another embodiment, the composition may include a conditioning agent, such as a dimethicone copolyol and/or other silicone-based surfactants. Suitable conditioning agents include polydimethylsiloxane polyether copolymers with pendant polyethylene oxide side chains, polydimethylsiloxane polyether copolymers with pendant polypropylene oxide side chains, polydimethylsiloxane polyether copolymers with pendant mixed polyethylene oxide and polypropylene oxide side chains, polydimethylsiloxane polyether copolymers with pendant mixed poly(ethylene)(propylene)oxide side chains, polydimethylsiloxane polyether copolymers with pendant organobetaine side chains, polydimethylsiloxane polyether copolymers with pendant carboxylate side chains, polydimethylsiloxane polyether copolymers with pendant quaternary ammonium side chains; and also further modifications of the preceding copolymers containing pendant (C2-C30) straight, branched, or cyclic alkyl moieties. Examples of commercially available dimethicone copolyols useful herein include PEG-10 Dimethicone available from Shin Etsu, Dow Corning® 190, 193, Q2-5220, 2501 Wax, 2-5324 fluid, and 5225 C (this latter material being sold as a mixture with cyclomethicone) available from Dow Corning Corporation. Cetyl dimethicone copolyol is commercially available as a mixture with polyglyceryl-4 isostearate (and) hexyl laurate and is sold under the tradename ABIL® WS-08 (also available from Goldschmidt). Other nonlimiting examples of suitable dimethicone copolyols include lauryl dimethicone copolyol, dimethicone copolyol acetate, dimethicone copolyol adipate, dimethicone copolyolamine, dimethicone copolyol behenate, dimethicone copolyol butyl ether, dimethicone copolyol hydroxy stearate, dimethicone copolyol isostearate, dimethicone copolyol laurate, dimethicone copolyol methyl ether, dimethicone copolyol phosphate, dimethicone copolyol sulfosuccinate and dimethicone copolyol stearate.

### EXAMPLE

The composition is illustrated by the following non-limiting example. The composition is prepared by mixing the following ingredients according to the amounts in Table I below:

TABLE I

| Component | Weight % |
|---|---|
| Cyclopentasiloxane¹ | 89.59 |
| High Molecular Weight Silicone Elastomer in Dimethicone² | 4.94 |
| High Molecular Weight Silicone Elastomer in | 4.42 |
| Cyclopentasiloxane³ | |
| Polyethylene glycol (PEG)-10 Dimethicone⁴ | 0.25 |
| Sodium Laneth 40 Maleate/Styrene Sulfonate Copolymer⁵ | 0.10 |
| Mango Melody⁶ | 0.50 |
| Argan Oil⁷ | 0.10 |
| Jojoba Seed Oil⁸ | 0.10 |

¹Available from Dow Corning under the trade name DC 245
²Available from Dow Corning under the trade name DC 9041 and described by Dow Corning as a dimethicone (and) dimethicone crosspolymer
³Available from Dow Corning under the trade name DC 9546 and described by Dow Corning as a Cyclopentasiloxane (and) dimethicone crosspolymer (and) dimethicone/vinyl dimethicone crosspolymer (and) dimethiconol
⁴Available from Shin Etsu under the trade name KF-6043
⁵Available from Croda under the trade name Mirustyle XHP
⁶Available from Belmay Fragrances
⁷Available from Charkit Chemical Corporation
⁸Available from Columbus Foods

In the example provided in Table I, batches, in either 1 or 2 pound quantities, of the blow dry treatment were prepared by individually adding 89.59% by weight cyclopentasiloxane (DC 245), 4.94% by weight of the silicone elastomer in dimethicone, and 4.42% by weight of the silicone elastomer in cyclopentasiloxane to a Caframo lab mixer having three blades with a total diameter of 2-2.5 inches. The ingredients were then blended. The mixing speed was increased incrementally so that the elastomers blended with the cyclopentasiloxane until smooth. The speed of the mixer was then reduced to prevent bubbles from forming.

In a separate, but similar mixer, the PEG-10 dimethicone and sodium laneth 40 maleate/styrene sulfonate copolymer were pre-blended. They were then added to the cyclopentasiloxane mixture to form the basic treatment composition.

Finally, 0.50% by weight mango melody fragrance, 0.10% by weight argan oil, and 0.10% by weight jojoba seed oil were added to the basic treatment composition to form the final treatment composition. In other embodiments, it is contemplated that one, all, or none of fragrance and oils may be included in the final treatment composition. It is also noted that the overall final treatment composition may include as much as, but no more than 1.0% water, by weight.

The final treatment composition prepared according to Table I was found to be capable of reducing the drying time of wet hair. For purposes of this disclosure, to “dry” wet hair means to remove at least 85% of added water weight. To be capable of reducing the drying time of wet hair, the composition decreases the time, in minutes, needed to evaporate from 85% to 100% of the added water weight on a swatch of human hair when subjected to a pre-selected amount of heat, as compared to a similar, but untreated, swatch of hair subjected to the same amount of heat.

The composition prepared according to Table I was found to be capable of reducing the drying time of wet hair by up to 50%, compared to untreated wet hair. More specifically, it is noted that each of samples A-E treated with the composition prepared according to Table I had reached a dry state within the first time interval of 15 minutes, whereas samples F-J did not reach a dry state until the second time interval of 30 minutes. Desirably, the composition will decrease the time required to remove about 85% to about 100% of added water weight by at least 35% when compared to the amount of time required to remove the same amount of water from hair not treated with the composition. In certain embodiments, the amount of time required to dry hair treated with the composition is decreased by at least 50%.

In order to compare the time needed to blow dry wet hair, the dry weight of several color treated hair swatches was measured. Each swatch, Samples A-J, was submerged in water and uniformly towel blotted to remove some amount of water. The towel-dried wet swatches were then weighed. Five pump sprays (0.45-0.5 g total) of the composition made according to the specifications of Table I were applied to Samples A-E using a spray bottle with a pump dimension of 37 MS and a 180 microliter dosage.

The samples were placed under a conventional hood drier set on perm, 120° F. (±2° F.), and allowed to dry for 15 minutes. All samples were then weighed. This procedure was repeated an additional three times, for a total of 60 minutes of time under the drier hood for all of the samples.

The water weight of all of the samples, excluding the starting “dry” weight of the swatches, is detailed below in Table II:

TABLE II
Water Weight (g)

| | Initial | 15 Min | 30 min | 45 min | 60 min |
|---|---|---|---|---|---|
| A | 1.4195 | 0.1619 | −0.2533 | −0.4796 | −0.5105 |
| B | 0.8753 | −0.0185 | −0.2508 | −0.4551 | −0.5096 |
| C | 0.6021 | −0.0608 | −0.2536 | −0.4281 | −0.4634 |
| D | 1.1299 | 0.0934 | −0.2114 | −0.4841 | −0.5434 |
| E | 1.1328 | 0.1229 | −0.1473 | −0.4267 | −0.4858 |
| F | 0.8954 | 0.4532 | 0.0943 | −0.0347 | −0.0316 |
| G | 0.8075 | 0.2276 | 0.0002 | −0.0388 | −0.0404 |
| H | 0.5972 | 0.1672 | 0.0023 | −0.0266 | −0.0243 |
| I | 0.5051 | 0.1354 | −0.0065 | −0.0233 | −0.0245 |
| J | 0.7594 | 0.2918 | 0.0071 | −0.0179 | −0.0277 |

It is noted that some of the samples were measured to have a negative weight after they were dried (as well as having lost more then 100% of the added water weight in Tables III and IV below). A certain amount of water is normally present in human hair, having been absorbed from the environment, the particular amount generally dependant on the relative humidity of the environment to which the hair has been exposed. Depending on the relative humidity of the environment, hair may increase in weight as much as 20% based upon absorption of water. For the purposes of testing, the humidity of the testing facility was kept constant during testing procedures. Therefore, the water present in the samples prior to drying was likely removed from the hair samples along with the water added for testing purposes when the hair was subjected to the hood dryers.

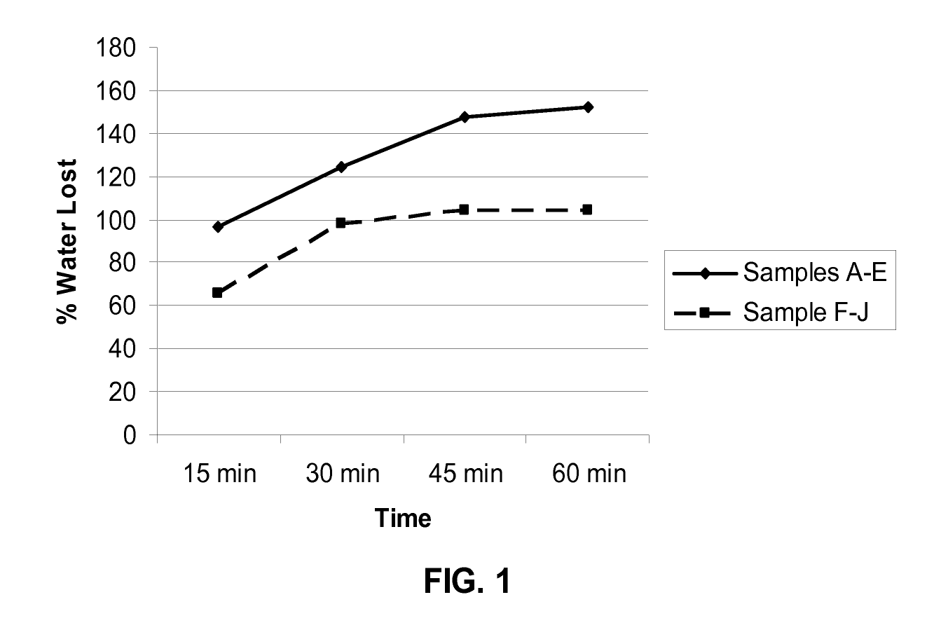

In addition to calculating the weight of water lost during the testing procedure, the weight percent of water loss for the treated samples (A-E) and untreated samples (F-J) was calculated. The weight percent of added water lost over the course of an hour is set forth in Table III below and the average weight percent lost over the course of an hour is set forth in Table IV and FIG. 1.

TABLE III
% Added Water Removed

| | Initial | 15 Min | 30 min | 45 min | 60 min |
|---|---|---|---|---|---|
| A | 0.0 | 89 | 100+ | 100+ | 100+ |
| B | 0.0 | 100+ | 100+ | 100+ | 100+ |
| C | 0.0 | 100+ | 100+ | 100+ | 100+ |
| D | 0.0 | 92 | 100+ | 100+ | 100+ |
| E | 0.0 | 89 | 100+ | 100+ | 100+ |
| F | 0.0 | 49 | 89 | 100+ | 100+ |
| G | 0.0 | 72 | 100 | 100+ | 100+ |
| H | 0.0 | 72 | 100 | 100+ | 100+ |
| I | 0.0 | 73 | 100+ | 100+ | 100+ |
| J | 0.0 | 62 | 99 | 100+ | 100+ |

TABLE III
Average % Water Loss per Time Interval
(based upon amount of water added by submerg-
ing hair swatch and blotting with towel)

| | 15 min | 30 min | 45 min | 60 min |
|---|---|---|---|---|
| Samples A-E | 96.34 | 124.06 | 147.48 | 152.42 |
| Sample F-J | 65.59 | 97.88 | 104.02 | 104.22 |

As demonstrated above, embodiments prepared according to the present disclosure decrease the time needed to dry wet hair by at least 50%. While the composition and methods herein have been described with a number of embodiments, the scope is not intended to be limited by the specific embodiments. Modifications and variations from the described embodiments exist. Although numerous ingredients suitable for formulating the hair styling composition have been listed, the list is by no means exhaustive.

1. (canceled)

2. (canceled)

3. A method for reducing the drying time of wet hair, comprising:
supplying a composition comprising a combination of about 89 to about 95 percent by weight of at least one volatile silicone compound, about 4 to about 10 percent by weight of at least one high molecular weight silicone elastomer, a thermoprotective agent, and a conditioning agent;
wherein the composition is capable of reducing a drying time of wet hair by at least 35 percent.

4. The method of claim 3, wherein the at least one volatile silicone compound comprises cyclopentasiloxane; and wherein the at least one high molecular weight silicone elastomer comprises about 4 to about 5 percent by weight of a dimethicone crosspolymer in dimethicone and about 0.5 to about 5 percent by weight of a mixture of a dimethicone crosspolymer, dimethicone/vinyl, dimethicone cross polymer, and dimethiconol in cyclopentasiloxane.

5. The method of claim 4, wherein the thermoprotective agent comprises about 0.1 to about 0.3 percent by weight of polyethylene glycol-10 dimethicone and the conditioning agent comprises about 0.1 to about 1.0 percent by weight of a sodium laneth 40-maleate/styrene sulfonate copolymer.

6. The method of claim 5, wherein the composition also includes an additive selected from one or more of a fragrance additive, argan oil, and jojoba seed oil.

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. A method for reducing the drying time of wet hair, comprising:
applying added water to human hair to obtain wet hair;
applying a nonaqueous composition comprising from about 85 percent to about 99 percent cyclopentasiloxane, a first silicone elastomer comprising about 0.5 to about 5 percent by weight of a mixture of a dimethicone crosspolymer, dimethicone/vinyl dimethicone crosspolymer, and dimethiconol, and a second silicone elastomer comprising about 4 to about 5 percent of a mixture of a dimethicone and cetearyl dimethicone crosspolymer to the wet hair;
applying heat to the wet hair; and
removing from about 85% to about 100%.

20. The method of claim 19 wherein the application of the nonaqueous composition reduces a drying time of the wet hair by at least 35% compared to a drying time of wet hair to which the nonaqueous composition is not applied.

21. The method of claim 21, wherein the nonaqueous composition further comprises an additive selected from one or more of fragrance, argan oil, and jojoba seed oil.

22. The method of claim 21, wherein the at least one volatile silicone compound comprises cyclopentasiloxane;
wherein the at least one high molecular weight silicone elastomer comprises about 4 to about 5 percent by weight of a dimethicone crosspolymer in dimethicone and about 0.5 to about 5 percent by weight of a mixture of a dimethicone crosspolymer, dimethicone/vinyl, dimethicone cross polymer, and dimethiconol in cyclopentasiloxane.

23. The method of claim 21, wherein the thermoprotective agent comprises about 0.1 to about 0.3 percent by weight of polyethylene glycol-10 dimethicone and the conditioning agent comprises about 0.1 to about 1.0 percent by weight of a sodium laneth 40-maleate/styrene sulfonate copolymer.

24. The method of claim 21, wherein heat is applied to the wet hair includes at about 120° F. for at least 15 minutes.
